(19) United States
(12) Patent Application Publication
(10) Pub. No.: US 2017/0332908 A1
(43) Pub. Date: Nov. 23, 2017

(54) COMMUNICATION DEVICE, COMMUNICATION METHOD, AND PROGRAM

(71) Applicant: Sony Corporation, Tokyo (JP)

(72) Inventor: Masahiro Uno, Tokyo (JP)

(21) Appl. No.: 15/527,543

(22) PCT Filed: Nov. 12, 2015
(86) PCT No.: PCT/JP2015/081825

§ 371 Date: May 17, 2017

(30) Foreign Application Priority Data
Nov. 25, 2014 (JP) 2014-237704

(51) Int. Cl. A61B 5/00; A61B 5/0488; H04B 5/02; A61B 5/0402; H04L 29/06

(52) U.S. Cl. CPC A61B 5/0028; H04B 5/02; A61B 5/0402; A61B 5/0488; H04L 63/0861

(57) ABSTRACT

The present technology relates to a communication device, a communication method, and a program that enable improvement in security of electric field communication. Biological information about a user is detected in accordance with an action of the user, and electric field communication being performed by an electric field communication unit is controlled in accordance with the biological information. The present technology can be applied to communication devices that perform electric field communication using an electric field, such as intra-body communication using the human body as a communication medium.

### TECHNICAL FIELD

The present technology relates to communication devices, communication methods, and programs, and more particularly, to a communication device, a communication method, and a program that enable improvement in security of electric field communication.

### BACKGROUND ART

Electric field communication using electric fields includes intra-body communication using the human body as the communication medium. In an intra-body communication system that performs such intra-body communication, when a user carrying an intra-body communication device to perform intra-body communication touches another intra-body communication device, communication starts between the intra-body communication device the user is carrying and the other intra-body communication device.

That is, when the user touches another intra-body communication device, a communication channel formed with the body of the user is established between the intra-body communication device of the user (the intra-body communication device the user is carrying) and the other intra-body communication device. Then, with the establishment of the communication channel serving as the trigger, communication starts between the intra-body communication device of the user and the other intra-body communication device.

As described above, in an intra-body communication system, communication starts when a user carrying an intra-body communication device touches another intra-body communication device. Therefore, even if the user has inadvertently or unintentionally touched another intra-body communication device though the user has no intention to conduct communication, communication starts between the intra-body communication device of the user and the other intra-body communication device.

In a case where intra-body communication starts between the intra-body communication device of the user and the other intra-body communication device though the user has no intention to conduct communication, if the personal information about the user is stored in the intra-body communication device of the user, for example, the personal information might be read out without the user noticing it. The personal information about the user being read out without the user noticing it is not preferable in terms of security, since the personal information is the name, the contact number, the address, a password, or the like of the user.

To counter this, a portable electric field communication device has been suggested. The portable electric field communication device senses a change in operation of the device, and becomes capable of electric field communication in a case where the change in operation matches authentication. information (see Patent Document 1, for example).

### CITATION LIST

### PATENT DOCUMENT

Patent Document 1: Japanese Patent Application Laid-Open No. 2010-074608

### SUMMARY OF THE INVENTION

### Problems to be Solved by the Invention

In a case where electric field communication is allowed when a change is operation of the device matches authentication information, if a complicated operation change is registered, it is troublesome to reproduce the operation change registered as the authentication information, and the user might forget the operation change.

If a simple operation change is registered as the authentication information, on the other hand, a third person might be able to easily guess the operation change as the authentication information, which is not preferable in terms of security.

The present technology has been developed in view of those circumstances, and is to readily enable improvement in security of electric field communication.

### Solutions to Problems

A communication device according to the present. technology is a communication device that includes: an electric field communication unit that performs electric field communication using an electric field; a sensor that detects biological information about the user, in accordance with an action of a user; and a control unit that controls the electric field communication being performed by the electric field communication unit, in accordance with the biological information.

A communication method according to the present technology is a communication method implemented by a communication device that includes: an electric field communication unit that performs electric field communication using an electric field; and a sensor that detects biological information about the user, in accordance with an action of a user. The communication method includes the step of controlling the electric field communication being performed by the electric field communication unit, in accordance with the biological information.

A program according to the present technology is a program to be executed by a computer that controls a communication device that includes: an electric field communication unit that performs electric field communication using an electric field; and a sensor that detects biological information about the user, in accordance with an action of a user. The program causes the computer to carry out the step of controlling the electric field communication being performed by the electric field communication unit, in accordance with the biological information.

In a communication device, a communication method, and a program according to the present technology, biological information about a user is detected in accordance with an action of the user, and electric field communication being performed by an electric field communication unit is controlled in accordance with the biological information.

It should be noted that the communication device may be an independent device, or may be internal blocks constituting a single device.

In addition, the program to be provided may be transmitted via a transmission medium or may be recorded on a recording medium.

### EFFECTS OF THE INVENTION

According to the present technology, it is possible to readily enable improvement in security of electric field communication.

It should be noted that effects of the present technology are not limited to the effect described above, and may include any of the effects described in the present disclosure.

### BRIEF DESCRIPTION OF DRAWINGS

FIG. 1 is a diagram for explaining intra-body communication using the human body as a communication medium.

FIG. 2 is a diagram showing an example of use of an intra-body communication system.

FIG. 3 is a plan view and a cross-sectional view of an example configuration of an embodiment of a wearable device to which the present technology is applied.

FIG. 4 is a block diagram showing an example electrical configuration of a main unit 41.

FIG. 5 is a diagram showing an example of use of a wearable device 40.

FIG. 6 is a flowchart for explaining an example process to be performed by the wearable device 40 and an intra-body communication device 100 on the other end of communication.

FIG. 7 is a block diagram showing another example electrical configuration of the main unit 41.

### MODES FOR CARRYING OUT THE INVENTION

<Intra-Body Communication>

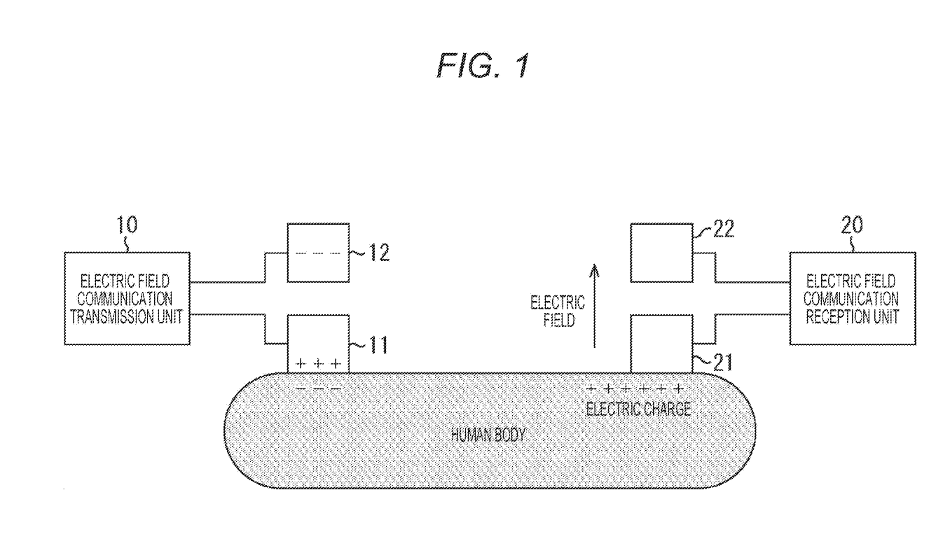

FIG. 1 is a diagram for explaining intra-body communication using the human body as a communication medium.

That is, FIG. 1 is a diagram showing an example configuration of an intra-body communication system that performs intra-body communication.

In FIG. 1, the intra-body communication system includes an electric field communication transmission unit 10 and an electric field communication reception unit 20.

The electric field communication transmission unit 10 includes two electrodes 11 and 12 (two poles), and transmits data through electric field communication using an electric field.

The electric field communication reception unit 20 includes the two poles of electrodes 21 and 22, and receives data through electric field communication using an electric field.

In the intra-body communication system having the above configuration, the electrode 11, which is one of the two poles of the electrodes 11 and 12 of the electric field communication transmission unit 10, is in contact with the human body serving as the communication medium. Likewise, the electrode 21, which is one of the two poles of the electrodes 21 and 22 of the electric field communication reception unit 20, is in contact with the human body serving as the communication medium.

The electric field communication transmission unit 10 applies a voltage between the two poles of the electrodes 11 and 12, the voltage corresponding to the current transmission target data. The human body is electrically charged in accordance with the voltage applied between the electrodes 11 and 12.

As the human body is electrically charged, an electric field corresponding to the current transmission target data is generated because of the charging. This electric field generates a voltage (a potential difference) between the two poles of the electrodes 21 and 22 of the electric field communication reception unit 20.

The electric field communication reception unit 20 detects and amplifies the voltage between the two poles of the electrodes 21 and 22, and determines the voltage, to restore the data.

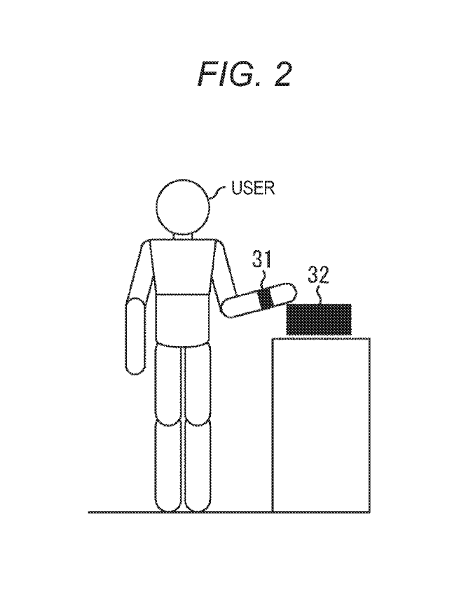

FIG. 2 is a diagram showing an example of use of an intra-body communication system.

The user is wearing a wristband 31 that is a wristwatch-type wearable device around his/her wrist, for example. The wearable device 31 carries an intra-body communication system similar to the intra-body communication system of FIG. 1, which includes the electric field communication transmission unit 10 and the electric field communication reception unit 20.

Like the wearable device 31, a stationary device 32 carries an intra-body communication system similar to the intra-body communication system of FIG. 1, which includes the electric field communication transmission unit 10 and the electric field communication reception unit 20. The stationary device 32 is installed (secured) in a predetermined position.

When the user wearing the wristband 31 touches the stationary device 32 (with his/her finger or the like), a communication channel formed with the body of the user is established between (the intra-body communication system of) the wristband 31 and (the intra-body communication system of) the stationary device 32.

With the establishment of the communication channel being the trigger, intra-body communication (electric field communication using the human body as the communication medium) starts between the wristband 31 and the stationary device 32. In this case, even if the user has inadvertently or unintentionally touched the stationary device 32 though the user has no intention to conduct communication, intra-body communication starts between the wristband 31 of the user and the stationary device 32.

In a case where intra-body communication starts between the wristband 31 of the user and the stationary device 32 though the user has no intention to conduct communication as above, if the personal information about the user is stored in the wristband 31 of the user, for example, the personal information might be read out without the user noticing it. The personal-information about the user being read out without the user noticing it is not preferable in terms of security.

Therefore, an operation unit such as buttons for controlling intra-body communication is provided on the wristband 31, for example. In a case where the operation unit is operated to perform intra-body communication, intra-body communication starts. In this manner, it is possible to prevent intra-body communication the user has no intention to start.

By the method in which an operation unit for controlling intra-body communication is provided on the wristband 31, however, intra-body communication is also performed in a case where a third party operates the operation unit. Intra-body communication being performed through an operation by a third party as a trigger as above is not preferable in terms of security.

<Embodiment of a Wearable Device to Which the Present Technology Is Applied>

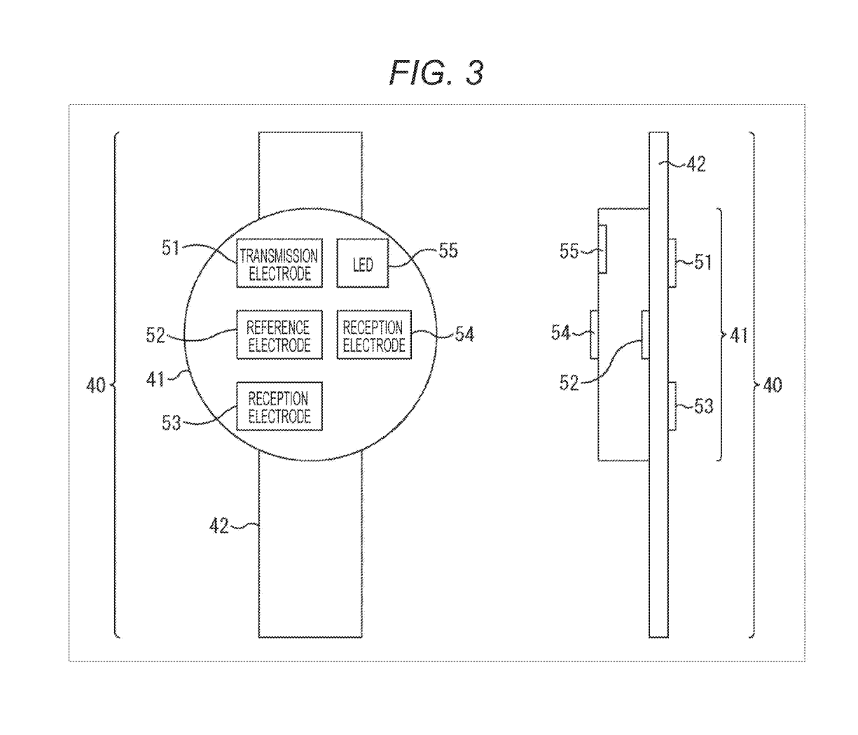

FIG. 3 shows a plan view of an example configuration. of an embodiment of a wearable device to which the present technology is applied, and a cross-sectional view of the right side surface of the wearable device.

In FIG. 3, a wearable device 40 is a wristwatch-type wearable device that is capable of electric field communication as intra-body communication, and is formed with a main unit 41 and a belt (a wristband) 42. As the belt 42 is wound around an arm (a wrist) of the user, for example, the wearable device 40 is attached to the user.

The main unit 41 includes a transmission electrode 51, a reference electrode 52, reception electrodes 53 and 54, and a light emitting diode (LED) 55.

It should be noted that, for easier understanding of the mounting of the transmission electrode 51 and the LED 55, all the components from the transmission electrode 51 to the LED 55 in FIG. 3 are shown as seen from front.

In the main unit 41, however, the transmission electrode 51, the reference electrode 52, and the reception electrode 53 are mounted so as not to be seen from front, and the reception electrode 54 and the LED 55 are mounted so as to be seen from front.

That is, the transmission electrode 51 and the reception electrode 53 are exposed through the back surface of the main unit 40 so as to be brought into contact with the body of the user when the user wears the wearable device 40.

It should be noted that the transmission electrode 51 and the reception electrode 53 do not need to be brought into contact with the body of the user when the user wears the wearable device 40. That is, when the user wears the wearable device 40, there may be a certain distance between the body of the user and each of the transmission electrode 51 and the reception electrode 53.

The reference electrode 53 is provided in the main unit 41 so as not to be brought into contact with the body of the user when the user wears the wearable device 40.

The reception electrode 54 is exposed through the front surface of the main unit 41, for example, so that the user can easily touch the reception electrode 54 when intending to touch it.

The LED 55 is exposed through the front surface of the main unit 41, for example, so that the user can easily see the LED 55.

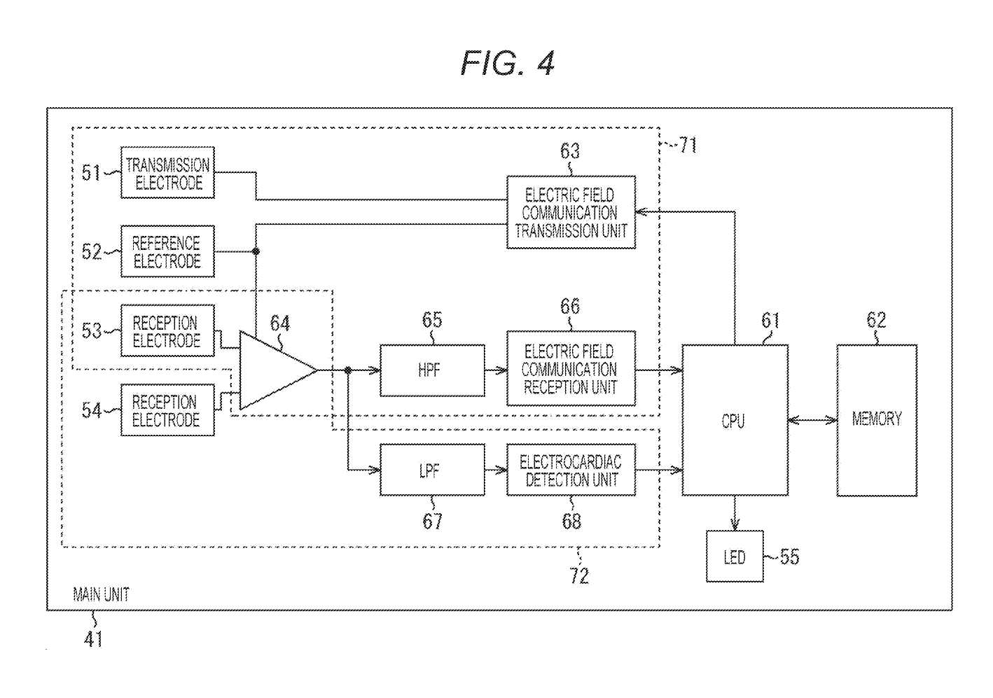

FIG. 4 is a block diagram showing an example electrical configuration of the main unit 41 of FIG. 3.

It should be noted that, in the drawing, the components equivalent to those in FIG. 3 are denoted by the same reference numerals as those used in FIG. 3, and explanation thereof is not repeated herein.

The main unit 41 includes not only the transmission electrode 51 through the LED 55, but also a central processing unit (CPU) 61, a memory 62, an electric field communication transmission unit 63, a differential amplifier 64, a high pass filter (HPF) 65, an electric field communication reception unit 66, a low pass filter (LPF) 67, and an electrocardiac detection unit 66.

The CPU 61 functions as a computer that controls the entire wearable device 40 and performs various other processes by executing a program stored in the memory 62.

That is, the CPU 61 controls intra-body communication as electric field communication being performed by the electric field communication transmission unit 63, in accordance with the user's electrocardiographic waveform supplied as the user's biological information from the electrocardiac detection unit 68, for example.

Specifically, the CPU 61 conducts authentication of the user, using the user's biological information supplied from the electrocardiac detection unit 68. If the authentication of the user is successful, the CPU 61 causes the electric field communication transmission unit 63 to start intra-body communication.

The CPU 61 then reads, from the memory 62, the current transmission target data through the intra-body communication, and supplies the data to the electric field communication transmission unit 63. In addition, in a case where data received through intra-body communication is supplied from the electric field communication reception unit 66, the CPU 61 also performs necessary processing, such as supplying and storing the data into the memory 62.

Further, in a case where a signal from the device on the other end of intra-body communication with the wearable device 40 is supplied from the electric field communication reception unit 66, the CPU 61 turns on the LED 55, in accordance with the signal.

That is, by turning on the LED 55, the CPU 61 prompts the user to act so that an electrocardiographic waveform as the biological information is detected by the electrocardiac detection unit 68. The aspect that the CPU 61 prompts the user's action by turning on the LED 55 will be described later in detail.

It should be noted that the program to be executed by the CPU 61 (a computer) may be recorded beforehand in the memory 61, or may be stored (recorded) in a removable recording medium to be provided and be then installed into the wearable device 40. The removable recording medium may be a flexible disk, a compact disc read only memory (CD-ROM), a magneto-optical (MO) disk, a digital versatile disc (DVD), a magnetic disk, or a semiconductor memory, for example.

In addition, the program can be installed into the wearable device 40 from. the removable recording medium, or can be downloaded and installed into the wearable device 40 via a communication network or a broadcasting network. That is, the program can be wirelessly transferred from a download site, for example, to the wearable device 40 via an artificial satellite for digital satellite broadcasting, or can be transferred by cable to the wearable device 40 via a network such as a local area network (LAN) or the Internet.

The memory 62 stores the program to be executed by the CPU 61, and the data necessary for the CPU 61 to operate. The memory 62 further stores the information necessary for the CPU 61 to conduct authentication using biological information. In addition to the above, the memory 62 stores personal information such as the user's name, as necessary.

The electric field communication transmission unit 63 applies a voltage between the transmission electrode 51 and the reference electrode 52, the voltage corresponding to the current transmission target data supplied from the CPU 61. By doing so, the electric field communication transmission unit 63 transmits the current transmission target data through electric field communication as intra-body communication.

Specifically, the electric field communication transmission unit 63 converts the current transmission target data into a baseband Manchester code, for example, and supplies a voltage corresponding to the baseband Manchester code between the transmission electrode 51 and the reference electrode 52.

Here, the baseband Manchester code is a code that assigns a rising edge and a falling edge to “0” and “1”, which are binary data as the current transmission target data. Such a baseband Manchester code characteristically contains no direct-current component in the signal spectrum, and is able to reduce low-frequency noise superimposed on a signal, where a differential decoding circuit is used for decoding (restoration.)

The intra-body communication scheme using baseband Manchester codes has been standardized as the “Close Capacitive Coupling Communication” system in ECMA-401 and ISO/IEC 17982.

As the electric field communication transmission unit 63 applies the voltage corresponding to the current transmission target data between the transmission electrode 51 and the reference electrode 52, the user's body in contact with the transmission electrode 51 is electrically charged. As the user's body is electrically charged, the current transmission target data is transmitted, with the user's body serving as the communication medium.

The differential amplifier 64 amplifies the voltage between the reception electrode 53 and the reference electrode 52, or the voltage between the reception electrodes 53 and 54, and supplies the voltage to the HPF 65 and the LPF 67.

The HPF 65 filters the voltage from the differential amplifier 64, to extract the high-pass signal of the voltage and supply the signal to the electric field communication reception unit 66.

The electric field communication reception. unit 66 restores the original data from the signal supplied from the HPF 65, and supplies the original data to the CPU 61.

The LPF 67 filters the voltage from the differential amplifier 64, to extract the low-pass signal of the voltage and supply the signal to the electrocardiac detection unit 68.

From the signal supplied from the LPF 67, the electrocardiac detection unit 68 detects an electrocardiographic waveform as a piece of the user's biological information, and supplies the electrocardiographic waveform to the CPU 61.

Here, the user touches the intra-body communication device on the other end of the communication to be performed by the wearable device 40. In a case where data is transmitted from the intra-body communication device as with the electric field communication transmission unit 63, the user's body is electrically charged in accordance with the data, and an electric field is generated. Then, with this electric field, a voltage (a potential difference) is then generated between the reception electrode 53 in contact with the user's body and the reference electrode 52 not in contact with the user's body.

The voltage between the reception electrode 53 and the reference electrode 52 is amplified by the differential amplifier 64, and is filtered by the HPF 65. As a result, a high-pass signal is extracted. At the electric field communication reception unit 66, the original data transmitted from the intra-body communication device on the other end of the communication is restored from the high-pass signal extracted by the HPF 65.

Also, in a case where the user wears the wearable device 40 around either the right arm or the left arm and touches the reception electrode 54 with a finger of the other arm, for example, the voltage generated between the reception electrodes 53 and 51 is supplied to the differential amplifier 64. That is, the voltage generated between the reception. electrode 53 in contact with one arm and the reception electrode 54 in contact with a finger of the other arm is supplied to the differential amplifier 64.

The voltage between the reception electrodes 53 and 54 is amplified by the differential amplifier 64, and is filtered by the LPF 67. As a result, a low-pass signal is extracted. From the low-pass signal extracted by the LPF 67, the electrocardiac detection unit 68 detects an electrocardiographic waveform as the biological information about the user.

It should be noted that, in the main unit 41 in FIG. 4, the transmission electrode 51, the reference electrode 52, the reception electrode 53, the electric field communication transmission unit 63, the differential amplifier 64, the HPF 65, and the electric field communication reception unit 66 constitute an electric field communication unit 71 that performs electric field communication as intra-body communication.

Also, in the main unit 41, the reception electrodes 53 and 54, the differential amplifier 64, the LPF 67, and the electrocardiac detection unit 68 constitute a sensor 72 that detects an electrocardiographic waveform as a piece of the user's biological information.

In a case where the user takes an action to touch the reception electrode 54, the sensor 72 detects an electrocardiographic waveform of the user. In view of this, the sensor 72 can be regarded as a sensor that detects an electrocardiographic waveform as biological information, in accordance with an action of the user (an action to touch the reception electrode 54).

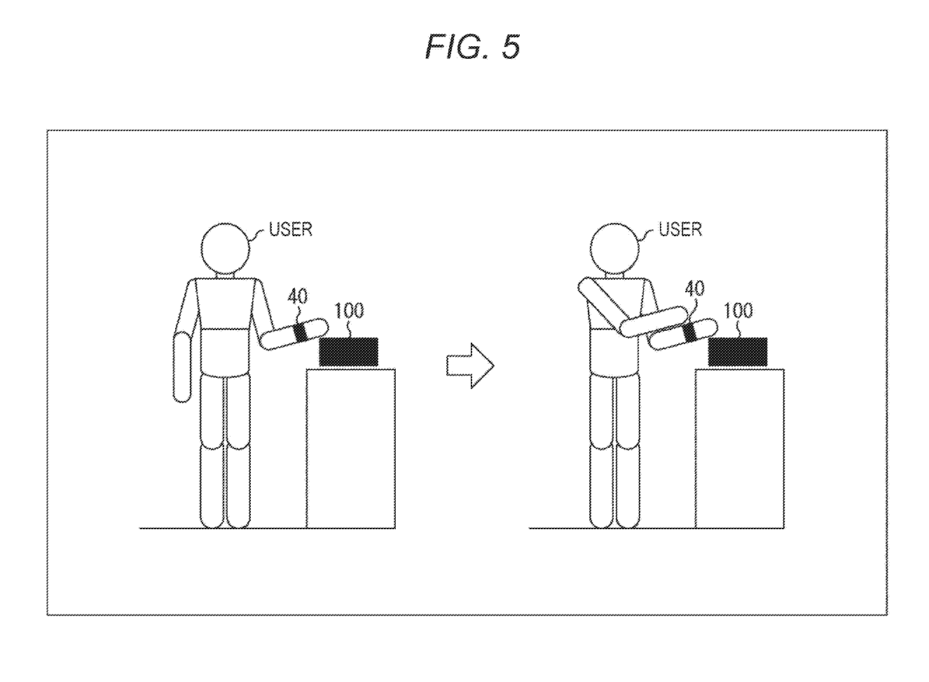

FIG. 5 is a diagram showing an example of use of the wearable device 40.

In FIG. 5, the user is wearing the wristwatch-type wearable device 40 around his/her left arm, for example. The user then touches a stationary intra-body communication device 100 with his/her left hand, the wearable device 40 being worn around his/her left arm. The intra-body communication device 100 is capable of electric field communication as intra-body communication.

As the user touches the intra-body communication device 100 with his/her left hand, a communication channel formed with the user's body is established between the wearable device 40 and the intra-body communication device 100.

In FIG. 2, establishment of a communication channel formed with the user's body between the wristband 31 and the stationary device 32 is the trigger for the wristband 31 and the stationary device 32 to start intra-body communication. On the other hand, the wearable device 40 does not start intra-body communication simply because a communication channel formed with the user's body is established between the wearable device 40 and the intra-body communication device 100.

When a communication channel formed with the user's body is established between the wearable device 40 and the intra-body communication device 100, the CPU 61 turns on the LED 55, to prompt the user to act so that an electrocardiographic waveform as biological information is detected by the sensor 72.

That is, as the CPU 61 turns on the LED 55, to prompt the user to taken an action to touch the reception electrode 54.

In a case where the user is prompted to touch the reception electrode 54 by switching on of the LED 55, the user touches the reception electrode 54 with a finger of his/her right arm, which is not the left arm around which the wearable device 40 is worn.

That is, since the user s wearing the wearable device 40 around his/her left arm and is touching the intra-body communication device 100 with his/her left hand, it is difficult for the user to touch the reception electrode 54 of the wearable device 40 with his/her left arm.

Therefore, the user touches the recon electrode 54 with a finger of his/her right arm, which is not the left arm around which the wearable device 40 is worn.

The voltage generated between the reception electrode 53 in contact with the left arm around which the wearable device 40 is worn and the reception electrode 54 in contact with the right hand is amplified by the differential amplifier 64 and is filtered by the LPF 67. The low-pass signal obtained through the filtering performed by the LPF 67 is then supplied from the LPF 67 to the electrocardiac detection unit 68, and the electrocardiac detection. unit 68 detects an electrocardiographic waveform of the user from the low-pass signal supplied from the LPF 67.

The electrocardiographic waveform detected by the electrocardiac detection unit 68 is supplied to the CPU 61.

The CPU 61 conducts authentication of the user, using the user's biological information supplied from the electrocardiac detection unit 68.

Specifically, in the memory 62, an electrocardiographic waveform as biological information about the user owning the wearable device 40 (an electrocardiographic waveform or the feature amount of an electrocardiographic waveform) is stored as the authentication information to be used in authenticating the user, for example.

The authentication information is registered (stored) into the memory 62 when the user is made to touch the reception electrode 54 during the initialization of the wearable device 40, for example.

In a case where the electrocardiographic waveform supplied from the electrocardiac detection unit 68 has the same features as those of the electrocardiographic waveform stored. as the authentication information in the memory 62, the CPU 61 determines the authentication of the user to be successful, and causes the electric field communication transmission unit 63 of the electric field communication unit 71 to start intra-body communication.

In a case where the electrocardiographic waveform supplied from the electrocardiac detection unit 68 does not have the same features as those of the electrocardiographic waveform stored as the authentication information in the memory 62, on the other hand, the CPU 61 determines the authentication of the user not to be successful, and does not allow the electric field communication transmission unit 63 to start intra-body communication.

In view of this, the wearable device 40 does not start intra-body communication only because the user touches the intra-body communication device 100 and a communication channel formed with the body of the user is established between the wearable device 40 and the intra-body communication device 100.

That is, the wearable device 40 does not start intra-body communication unless the user takes an action to touch the reception electrode 54 of the wearable device 40 though touching the intra-body communication device 100.

As a result, it is possible to prevent intra-body communication between the wearable device 40 and the intra-body communication device 100, and readout of the personal information stored in the memory 62, when the user touches the intra-body communication device 100 even though having no intention to conduct intra-body communication.

In addition, even when the user touches the intra-body communication device 100 and touches the reception electrode 54 of the wearable device 40, the wearable device 40 does not start intra-body communication unless the user authentication using the electrocardiographic waveform detected through the contact with the reception electrode 54 is successful.

Because of the above, even if a third person wears the wearable device 40 and touches the reception electrode 54, intra-body communication is not started between the wearable device 40 and the intra-body communication device 100. Thus, it is possible to prevent readout of the personal information stored in the memory 62.

As described above, the wearable device 40 does not start intra-body communication unless an authenticated user (the authentication of the user being successful) wears the wearable device 40 and takes an action to touch the reception electrode 54. Thus, security of intra-body communication can be readily improved.

Here, the wearable device 40 performs a process to turn on the LED 55 as an action facilitating process to prompt the user to act so that an electrocardiographic waveform as the biological information is detected by the electrocardiac detection unit 68. As the LED 55 is turned on in this action facilitating process, the user takes an action to touch the reception electrode 54 (this action will be hereinafter also referred to as the touch action). Through this touch action, an electrocardiographic waveform of the user is detected. If the authentication using the electrocardiographic waveform is successful, intra-body communication is started.

Therefore, the touch action can be regarded as display of the user's intention to conduct intra-body communication.

It should be noted that, where electrodes are brought into contact with portions at two points located on opposite sides of the heart, an electrocardiographic waveform can be detected with a higher degree of accuracy than in a case where electrodes are brought into contact with portions at two points located not on opposite sides of the heart. In the wearable device 40, the reception electrode 53 is in contact with the arm around which the wearable device 40 is worn, and the reception electrode 54 is in contact with the other arm. Therefore, the reception electrodes 53 and 54 are brought into portions at two points located on opposite sides of the heart. Thus, a highly accurate electrocardiographic waveform can be detected.

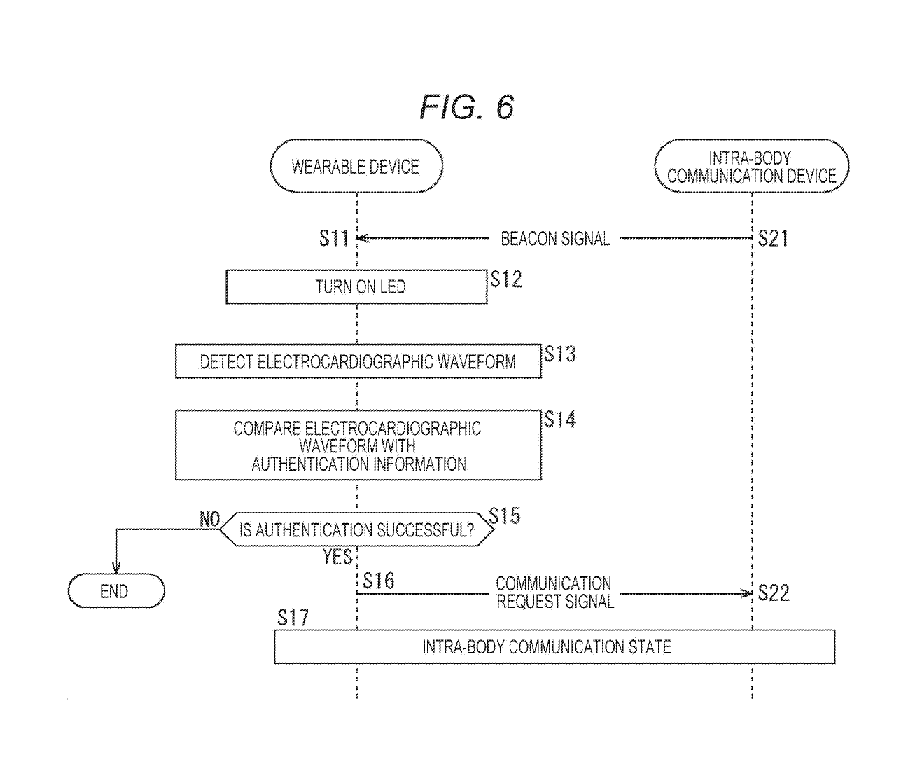

FIG. 6 is a flowchart for explaining an example process to be performed by the wearable device 40 shown in FIG. 5 and the intra-body communication device 100 on the other end of communication.

When the user wearing the wearable device 40 touches the intra-body communication device 100, a communication channel formed with the user's body is established between the wearable device 40 and the intra-body communication device 100.

As a communication channel formed with the user's body is established between the wearable device 40 and the intra-body communication device 100, the intra-body communication device 100 transmits a beacon signal in step S21.

The beacon signal is received by the wearable device 40 via the user's body in step S11.

Specifically, in the wearable device 40, the beacon signal is received by the electric field communication reception unit 66 via the reference electrode 52, the reception electrode 53, the differential amplifier 64, and the HPF 65, and is then supplied to the CPU 61.

As the beacon signal is supplied from the electric field communication reception unit 66, the CPU 61 turns on the LED 55 in step S12, to prompt the user to take a touch action.

When the user takes a touch action as the LED 55 is turned on, or when the user touches the reception electrode 54, the voltage generated between the reception electrode 53 and the reception electrode 54 is supplied to the electrocardiac detection unit 68 via the differential amplifier 64 and the LPF 67, and an electrocardiographic waveform of the user is detected in step S13.

The electrocardiographic waveform detected by the electrocardiac detection unit 68 is supplied to the CPU 61.

In step S14, the CPU 61 conducts authentication of the user by comparing the user's electrocardiographic waveform supplied from the electrocardiac detection unit 68 with the electrocardiographic waveform stored as the authentication information in the memory 62.

Then, in step S15, the CPU 61 determines whether the authentication of the user is successful.

If the CPU 61 determines the authentication of the user not to be successful in step S15, or if the user's electrocardiographic waveform supplied from the electrocardiac detection unit 68 do not have the same features as those of the electrocardiographic waveform stored as the authentication information in the memory 62, the wearable device 40 ends the process.

In this case, intra-body communication will not be performed thereafter between the wearable device 40 and the intra-body communication device 100.

If the CPU 61 determines the authentication of the user to be successful in step S15, or if the user's electrocardiographic waveform supplied from the electrocardiac detection unit 68 has the same features as those of the electrocardiographic waveform stored as the authentication information in the memory 62, on the other hand, the process moves on to step S16.

In step S16, the CPU 61 controls the electric field communication transmission unit 63, to transmit a communication request signal to the intra-body communication device 100 through intra-body communication, the communication request signal being a request for intra-body communication.

In step S22, the intra-body communication device 100 receives the communication request signal transmitted from the electric field communication transmission unit 63 of the wearable device 40.

In step S17, the wearable device 40 that has transmitted the communication request signal and the intra-body communication device 100 that has received the communication request signal enter a state where intra-body communication can be performed, and thus start intra-body communication.

As described above, in the wearable device 40, intra-body communication becomes possible when the user touches the intra-body communication device 100 and further takes a touch action to touch the reception electrode 54 to display an intention to conduct intra-body communication.

Because of this, the wearable device 40 can perform intra-body communication after confirming that the user has an intention to conduct intra-body communication.

Further, the wearable device 40 becomes capable of intra-body communication in a case where user authentication using the electrocardiographic waveform detected through the user's touch action is successful.

Thus, misconduct such as identity theft can be prevented.

Also, while the user is wearing the wearable device 40, the reception electrode 53 of the wearable device 40 is in contact with the user's body. When the user touches the reception electrode 54, an electrocardiographic waveform is detected from the voltage generated between the reception electrodes 53 and 54 in contact with the user, and authentication of the user is conducted.

Because of this, when the user is not wearing the wearable device 40, any electrocardiographic waveform is not detected even if the user touches the reception electrode 54, and authentication of the user is not conducted. Thus, intra-body communication can be prevented.

Further, when the wearable device 40 receives a beacon signal, the LED 55 is turned on in accordance with the beacon signal, and the user is prompted to take a touch action so that an electrocardiographic waveform is detected by the sensor 72.

Thus, the user can recognize a start of intra-body communication with the trigger being a touch action, as the LED 55 is turned on.

It should be noted that, although the sensor 72 detects an electrocardiographic waveform in FIG. 4, the sensor 72 may be a sensor that detects an electromyographic waveform that is not an electrocardiographic waveform, and the electromyographic waveform detected by the sensor can be used in authenticating the user.

Further, the sensor 72 may be a sensor that detects biological information excluding an electromyographic waveform, such as body temperature, perspiration, or blood pressure, and the biological information detected by the sensor can be used in authenticating the user.

Furthermore, the biological information to be detected by the sensor 72 is not limited to one kind of biological information. That is, the sensor 72 may detect multiple kinds of biological information, and the multiple kinds of biological information can be used in authentication the user.

Also, the sensor 72 may be not only a sensor that detects biological information about the user, but also a microphone that detects voice of the user or a camera that captures an image of the user's face. The biological information about the user, and the voice or the image of the user can be used in authenticating the user.

Also, the sensor 72 may be not only a sensor that detects biological information about the user, but also a sensor that detects movement of the user. The biological information about the user and the movement of the user can be used in authenticating the user. That is, in a case where the biological information and the movement of the user match the biological information and the movement registered beforehand as the authentication information, intra-body communication can be started.

Further, the wearable device 10 turns on the LED 55, to prompt the user to take a touch action. However, the measure to prompt the user to take a touch action is not limited to turning on the LED 55.

Specifically, the measure to prompt the user to take a touch action is not limited to turning on the LED 55, and may be outputting predetermined sound, or outputting a message in the form of an image or sound to prompt the user to take a touch action, for example.

<Another Example Configuration of the Main Unit 41>

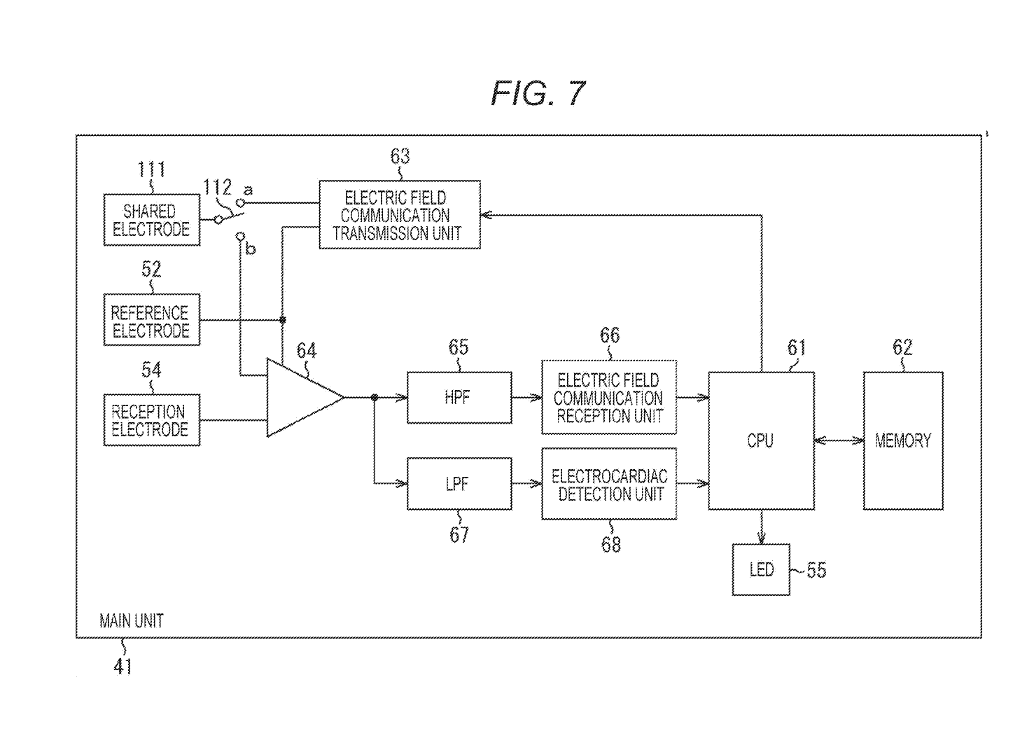

FIG. 7 is a block diagram showing another example electrical configuration of the main unit 41 of the wearable device 40.

It should be noted that, in the drawing, the components equivalent to those in FIG. 4 are denoted by the same reference numerals as those used in FIG. 4, and explanation thereof is not repeated herein.

The main unit 41 in FIG. 7 is the same as that in the case shown in FIG. 4 in including a reference electrode 52, a reception electrode 54, an LED 55, a CPU 61, and an electrocardiac detection unit 68.

However, the main unit 41 in FIG. 7 differs from that in the case shown in FIG. 4 in that the transmission electrode 51 and the reception electrode 53 are replaced with a shared electrode 111, and a switch 112 is newly added.

In FIG. 7, the shared electrode ill serves as both the transmission electrode 51 and the reception electrode 53 shown in FIG. 4. As the switch 112 is operated, the shared electrode 111 functions as the transmission electrode 51 or the reception electrode 53.

The switch 112 is connected to the shared electrode 111. A terminal a of the switch 112 is connected to the electric field communication transmission unit 63, and a terminal b of the switch 112 is connected to the differential amplifier 64.

Under the control of the CPU 61, the switch 112 is operated. to switch the terminal a or b.

In a case where the switch 112 selects the terminal a, the shared electrode 111 is connected to the electric field communication transmission unit 63 via the switch 112. In addition, in a case where the switch 112 selects the terminal b, on the other hand, the shared electrode 111 is connected to the differential amplifier 64.

In the main unit 41 having the above configuration, in a case where data is to be transmitted through intra-body communication, the switch 112 is switched to the terminal a As the switch 112 is switched to the terminal a, the shared electrode 111 and the electric field communication transmission unit 63 are connected to each other via the switch 112, and thus, the shared electrode 111 functions as the transmission electrode 51 shown in FIG. 4.

In a case where data is to be received through intra-body communication, and in a case where an electrocardiographic waveform is detected as biological information, on the other hand, the switch 112 is switched to the terminal b. As the switch 112 is switched to the terminal b, the shared electrode 111 and the differential amplifier 54 are connected to each. other via the switch 112, and thus, the shared electrode 111 functions as the reception electrode 53 shown in FIG. 4.

It should be noted that, like the transmission electrode 51 and the reception electrode 53 in FIG. 3, the shared electrode 111 in the wearable device 40 is located at a portion to be brought into contact with the user when the user wears the wearable device 40.

In this specification, the processes to be performed by a computer (the CPU 61) in accordance with a program are not necessarily performed in chronological order compliant with the sequence shown in the flowchart. That is, the processes to be performed by the computer in accordance with the program include processes to be performed in parallel or independently of one another (such as parallel processes or object-based processes).

In addition, the program may be executed by one computer, or may be executed in a distributive manner by more than one computer.

Furthermore, in this specification, a system means an assembly of components (devices, modules (parts), and the like) , and not all the components need to be provided in the same housing. In view of this, devices that are housed in different housings and are connected to each other via a network form a system, and one device having modules housed in one housing is also a system.

It should be noted that embodiments of the present technology are not limited to the above described embodiments, and various modifications may be made to them without departing from the scope of the present technology.

For example, the respective steps described with reference to the above described flowchart can be carried out by one device or can be shared among devices.

Further, in a case where more than one process is included in one step, the processes included in the step can be performed by one device or can be shared among devices.

In addition, the present technology can be applied not only to intra-body communication that is electric field communication using the human body as a communication medium, but also to electric field communication using electric fields.

Further, the advantageous effect described in this specification is merely an example, and the advantageous effects of the present technology are not limited to it and may include other effects.

It should be noted that the present technology may also be embodied in the configurations described. below.

<1>

A communication device including:

an electric field communication unit that performs electric field communication using an electric field;

a sensor that detects biological information about the user, in accordance with an action of a user; and

a control unit that controls the electric field communication being performed. by the electric field communication unit, in accordance with the biological information.

<2>

The communication device of <1>, in which the control unit conducts authentication of the user using the biological information, and, in a case where the authentication of the user is successful, the control unit causes the electric field communication unit to start the electric field communication.

<3>

The communication device of <1>or <2>, in which the sensor detects an electromyographic waveform.

<4>

The communication device of <3>, in which the sensor detects an electrocardiographic waveform.

<5>

The communication device of any of <1>to <4>, in which:

the sensor further detects movement of the user; and

the control unit controls the electric field communication being performed by the electric field communication unit, in accordance with the biological information and the movement of the user.

<6>

The communication device of any of <1>to <5>, in which, in accordance with a signal from a device on the other end of communication, the control unit prompts the user to act to cause the sensor to detect the biological information about the user.

<7>

The communication device of any of <1>to <6>, in which the electric field communication unit performs intra-body communication as the electric field communication, with a human body being a communication. medium.

<8>

The communication device of any of <1>to <7>, which is a wearable device.

<9>

A communication method implemented by a communication device that includes:

an electric field communication unit that performs electric field communication. using an electric field; and

a sensor that detects biological information about the user, in accordance with an action of a user,

the communication method including the step of controlling the electric field communication being performed by the electric field communication unit, in accordance with the biological information.

<10>

A program to be executed by a computer that controls a communication device that includes:

an electric field communication unit that performs electric field communication using an electric field; and

a sensor that detects biological information about the user, in accordance with an action of a user,

the program causing the computer to carry out the step of controlling the electric field communication being performed by the electric field communication unit, in accordance with the biological information.

### REFERENCE SIGNS LIST

10 Electric field communication transmission unit.

### 11, 12 Electrode

20 Electric field communication reception unit

### 21, 22 Electrode

### 31 Wristband

32 Stationary device
40 Wearable device
41 Main unit

### 42 Belt

51 Transmission electrode
52 Reference electrode
53, 54 Reception electrode

### 55 LED

### 61 CPU

### 62 Memory

63 Electric field communication transmission unit
64 Differential amplifier

### 65 HPF

66 Electric field communication reception unit

### 67 LPF

68 Electrocardiac detection unit
71 Electric field communication unit

### 72 Sensor

100 Intra-body communication device
111 Shared electrode

### 112 Switch

1. A communication device comprising:
an electric field communication unit configured to perform electric field communication using an electric field;
a sensor configured to detect biological information about the user, in accordance with an action of a user; and
a control exit configured to control the electric field communication being performed by the electric field communication unit, in accordance with the biological information.

2. The communication device according to claim 1, wherein the control unit conducts authentication of the user using the biological information, and, in a case where the authentication of the user is successful, the control unit causes the electric field communication unit to start the electric field communication.

3. The communication device according to claim 2, wherein the sensor detects an electromyographic waveform.

4. The communication device according to claim 3, wherein the sensor detects an electrocardiographic waveform.

5. The communication device according to claim 1, wherein:
the sensor further detects movement of the user; and
the control unit controls the electric field communication being performed by the electric field communication unit, in accordance with the biological information and the movement of the user.

6. The communication device according to claim 1, wherein, in accordance with a signal from a device on the other end of communication, the control unit prompts the user to act to cause the sensor to detect the biological information about the user.

7. The communication device according to claim 1, wherein the electric field communication unit performs intra-body communication as the electric field communication, with a human body being a communication medium.

8. The communication device according to claim 1, which is a wearable device.

9. A communication method implemented by a communication device, the communication device including:
an electric field communication unit configured to perform electric field communication using an electric field; and
a sensor configured to detect biological information about the user, in accordance with an action of a user,
the communication method comprising
the step of controlling the electric field communication being performed by the electric field communication unit, in accordance with the biological information.

10. A program to be executed by a computer that controls a communication device, the communication device including:
an electric field communication unit configured to perform electric field communication using an electric field; and
a sensor configured to detect biological information about the user, in accordance with an action of a user,
the program causing the computer to carry out
the step of controlling the electric field communication being performed by the electric field communication unit, in accordance with the biological information.
